(19) United States
(12) Patent Application Publication
(10) Pub. No.: US 2005/0181521 A1
(43) Pub. Date: Aug. 18, 2005

(54) Sampling and assay device together with methods for use thereof

(75) Inventors: Aimo Niskanen, Espoo (FI); Mika Saramaki, Karkkila (FI)

Correspondence Address: FOLEY & LARDNER, P.O. BOX 80278, SAN DIEGO, CA, 92138-0278, US

(73) Assignee: Ani Biotech Oy

(21) Appl. No.: 10/919,810

(22) Filed: Aug. 17, 2004

(30) Foreign Application Priority Data
Feb. 18, 2004 (EP) 04003603.0

(51) Int. Cl. G01N 33/558; G01N 33/543

(52) U.S. Cl. 436/514

(57) ABSTRACT

An immunochemical sampling device, kits including the sampling device, processes for production of the sampling device, and methods for use of the sampling device in lateral flow immunoassays. The sampling device comprises a solid support that is partially wrapped in a porous carrier, then covered in a hydrophobic cover. At its distal end, the porous carrier comprises a labeled binding reagent that is retained in the solid support until released into controlled flow with the liquid sample when the sampling device is brought into contact with a test strip.

### CROSS-REFERENCE TO RELATED PATENT APPLICATIONS

This application claims the benefit of the priority of European Patent Application No. 04003603.0, filed on Feb. 18, 2004.

### BACKGROUND OF THE INVENTION

The present invention relates to an immunochemical sampling device, especially to devices for conducting immunoassays that are suitable for home use, at doctors' offices and/or by technically untrained staff, involving a minimal level of skill from the users.

Methods and devices based on immunodiffusion are known from for example U.S. Pat. Nos. 4,757,002, 3,990,852 and 4,562,147. Immunochromatographic methods based on lateral flow are known from EP 0 291 194; EP 0 284 232; EP 0 250 137; U.S. Pat. Nos. 5,250,412; 5,084,245; 5,760,315; 6,375,896 and WO 86/03839.

U.S. Pat. No. 4,562,147 provides a radial immunodiffusion enzyme assay method for testing of pseudorabies antibodies in swine and other animals. Agar test plates are provided including an underlying adherent coating of solubilized non-infectious swine pseudorabies antigen. The result of the test is obtained from the diameters of the resulting colored zones which correlate with the titers obtained by the official virus neutralization test.

EP 0 291 194 relates to assays involving specific binding, especially immunoassays and devices therefor. The analytical test device disclosed comprises a hollow casing, containing a dry porous carrier, which communicates indirectly with the exterior of the casing via a bibulous sample receiving member. The carrier contains in a first zone a labeled specific binding reagent and in a second zone (test zone) spatially distinct from the first zone an unlabelled specific binding reagent for the same analyte. In use, the sample solution is applied directly to the test device when the test is performed, which exposes the test zone to risk of overflow.

EP 0 284 232 provides a solid phase assay for determining the presence or absence of analyte in a liquid sample. A test strip of the invention has a tracer movably supported on a first portion and a binder immobilized on a second portion (test zone). In use, the sample solution is applied directly to the test device when the test is performed, which exposes the test zone to risk of overflow.

EP 0 250 137 describes an immunoassay using colloidal gold for detecting a ligand in a sample, where a membrane strip is contacted with a sample and simultaneously or successively with a liquid reagent containing a ligand binding partner or ligand labeled with colloidal gold. To perform the test, the user must separately apply the liquid reagent containing the labeled ligand to the membrane strip.

U.S. Pat. No. 5,250,412 describes a device, method and kit for collecting and analyzing an analyte. The sample is collected with a swab, to which the user must also apply a separate liquid containing a labeled component, then a wash solution.

U.S. Pat. No. 5,084,245 provides a device for conducting diagnostic procedures based on immunological reactions using specimens gathered up in the absorbent tip of a swab. When used the swab is pushed through a passageway towards a sensitive element containing the necessary reagents for the test. Ribs are positioned in the passageway to squeeze the tip and express fluid of the swab. The results are visually observable by removing a guide member from the base component to uncover the sensitive element.

U.S. Pat. No. 5,760,315 provides a device for collection of a sample using a absorbing pad. The pad serves as a fluid reservoir for a volume of sample fluid sufficient to serve as a washing agent to remove excess labeled binding reagent not bound in the test zone of a corresponding test strip. A complex array of materials of varying hydrophobicity and porosity are utilized in the device to direct sample fluid, during its initial collection, away from labeled reagent disposed on or near the absorbing pad.

U.S. Pat. No. 6,375,896 describes a swab analyzer for the immunochemical detection of substances. A swabbing pen of the swab analyzer is used for the collecting of the sample. The sample must be eluted from the device using a separate elution liquid.

WO 86/03839 illustrates a solid phase diffusion assay where the sample is first mixed with a labeled binding substance and then applied to a region of a support with immobilised adsorbent molecules and allowed to diffuse therein. The diffusion pattern is visualized and measured.

It is evident that a variety of different test kits for performing immunoassays are available in the art. For home use in particular, many such devices enable sample collection and testing to be performed by the user in “one step” by allowing sample fluid to be directly applied to the test device; e.g., by placing a portion of the device into a urine stream. However, when a single component is used both to collect and test a fluid sample there is a risk that the sensitive reagents and the structure of the analytical device is will be contaminated or otherwise disturbed.

The present invention provides an improved two-component immunoassay device which enable the user to easily collect a liquid sample then transfer it to a test device for performance of the assay without risk of spillage or contamination.

### SUMMARY OF THE INVENTION

The present invention provides an immunochemical sampling device, kits and methods for use of the sampling device in lateral flow immunoassays. The device comprises a solid support that is partially wrapped in a porous carrier, then covered in a substantially impermeable hydrophobic cover. At its distal end, the porous carrier comprises a labeled binding reagent (e.g., an antibody or antigen) that is released from the porous carrier into controlled flow with the liquid sample when the sampling device is brought into contact with (e.g., pressed upon) a test strip element of an analyzer device.

In use, the wrapped portion of the sampling device is brought into contact with a fluid sample (e.g., by immersion or introduction into a stream of sample) so sample contacts the proximal edge of the porous carrier, which proximal edge is exposed beneath the hydrophobic cover. The porous carrier is wrapped around the solid support (which serves at its unwrapped end as a handle) in a manner designed to optimize flow of sample into the porous carrier, as well as flow of sample mixed with labeled binding reagent out of the porous carrier on contact with an analyzer device. The volume and rate of flow of sample through the porous carrier are controlled as well by pressure equilibrization means provided in the hydrophobic cover.

In particular, once sample fluid has been collected in the sampling device (where it mixes with labeled binding reagent), it is retained until released into the analyzer device by bringing the distal end of the sampling device into contact with the test strip of the analyzer device. Flow of sample fluid out of the sampling device occurs via diffusion or capilliary action mediated by contact with the test strip, and may be encouraged mechanically (e.g., by application of gentle pressure to the sampling device).

Preferably, the analyzer device comprises a test strip on which a binding reagent is immobilized within a test zone downstream of a sample application site at which no binding reagent need be bound. Most preferably, the analyzer device further comprises a solid housing for the test strip having a depression into which the sample application site of the test strip is deflected under gentle pressure applied to the sampling device, to facilitate flow of sample out of the porous carrier.

The analytes to be detected can be antigens of or antibodies against bacteria, virus, fungi and parasites or components and products thereof (including disease specific antibodies; e.g., antibodies against Helicobacter pylori, Hepatitis A, HIV_{1,2}, respiratory disorders, etc.); antigens excreted in urine (including luteinizing hormone (LH), follicle stimulating hormone (FSH) and human chorionic gonadotropin (hCG)); or antigens indicative of the presence of a narcotic or narcotic metabolite in a fluid sample.

The test system is easy to use, and may therefore be provided for at home use. Because sample fluid is not applied directly to the analyzer device, saturation of the test strip and/or contamination of the test zone are avoided. In short, the invention provides a device for collecting and testing sample fluids for the presence of analytes that is uniquely simple to use and manufacture.

### BRIEF DESCRIPTION OF THE DRAWINGS

FIG. 1 is a perspective view of a sampling device in accordance with the invention;

FIG. 2 is a close up perspective view of a covered porous carrier in accordance with the invention;

FIG. 3 is a perspective view of a sampling device in accordance with the invention, with its protective cover removed;

FIG. 4 is a side view of the sampling device of the invention and an analyzer device for use therewith;

FIG. 5 is a view seen from above of an analyzer device for use in detection of multiple analytes.

### DETAILED DESCRIPTION OF THE PREFERRED EMBODIMENTS

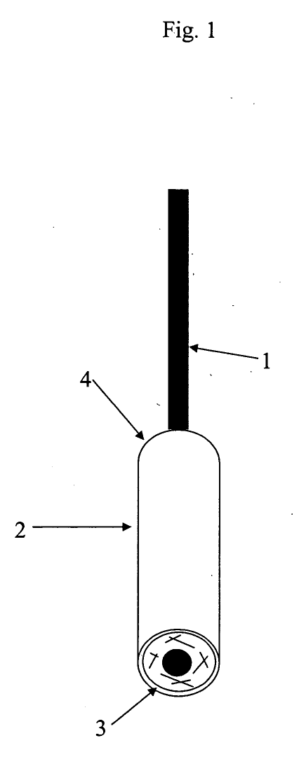

Turning to FIG. 1, a solid support 1 is shown in the form of a round stick, although those of ordinary skill in the art will appreciate that the solid support may be formed in any shape having opposing proximal and distal ends, around which a porous material may be wrapped. For example, solid support 1 can be an elongated round, flat or planar stick, which is solid or hollow.

A porous carrier 2 is disposed around the distal end of solid support 1, and is partially covered by hydrophobic cover 3. As shown in FIG. 1, porous carrier 2 is covered axially by hydrophobic cover 3, leaving only the surfaces at its opposing proximal end 4 and its distal end 5 exposed.

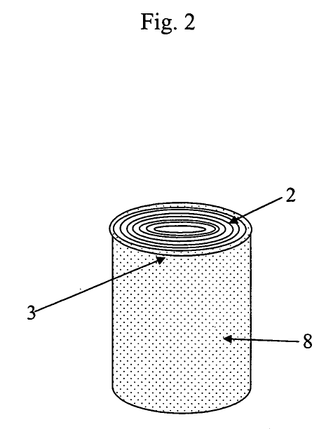

As shown in FIG. 2, porous carrier 2 comprises one or more layers, preferably 1 to 5 layers of a porous material. The porous material is selected from a group of materials consisting of paper, glass fiber, nylon, polyester or cellulose and derivatives thereof, most preferably the bibulous and/or hydrophilic forms of these materials. Conveniently, the porous material is nitrocellulose or a nonwoven polyester of variable porosity, and is blocked to render it inert to non-specific binding of proteins.

The blocking solution for making the porous material inert may be any conventional blocking solution. For example, the blocking solution may be a mixture comprising natural or synthetic polymers such as albumin (BSA, Bovine serum albumin), PEG (polyethylene glycol), PVA (polyvinyl alcohol) and PVP (polyvinyl pyrrolidone), nonionic detergents such as HEXA (hexane sulphonic. acid) and TRITON-X-100, SDS, BRIJ and preservation agents such as sugar, for example glucose, sucrose and trehalose or derivatives thereof.

Hydrophobic cover 3 is wrapped around the portion of porous carrier 2 to be covered, and secured thereon; e.g., with an inert adhesive. Hydrophobic cover 3 is substantially hydrophobic and impermeable, although it may be hydrophilic at its distal and/or proximal edges. Conveniently, hydrophobic cover 3 may be comprised of a clear polyester tape, mylar film, or other impermeable material. Optionally, a hydrophobic printed paper (not shown) having symbols indicative of, for example, instructions for collection of a sample and the identity of analytes that can be bound by the labeled binding reagents, may be disposed between hydrophobic cover 3 and porous carrier 2.

As further shown in FIG. 2, hydrophobic cover 3 is provided with a plurality of small vents 8 therethrough having a maximum diameter each of approximately 0.1 mm to 0.5 mm distributed over the 25 mm×51 mm piece of tape used to form hydrophobic cover 3. Preferably, vents 8 are provided across 1 to 50% of the surface area of hydrophobic cover 3, and most preferably between 10 and 25% of the surface area of hydrophobic cover 3.

Vents 8 serve to control the volume and rate of fluid flow through porous carrier 2. Although the invention is not to be limited to the mechanism by which vents 8 control fluid flow in the sampling device, it is believed that the vents serve to equalize the pressure within and without porous carrier 2 as sample fluid is applied thereto, thereby avoiding the formation of a pressure gradient along which fluid flow would be encouraged at the risk of washing out of labeled binding reagent. In operation, the vents serve to retain sample fluid in porous carrier 2 until the sampling device is placed into fluid communicative contact the test strip element of an analyzer device.

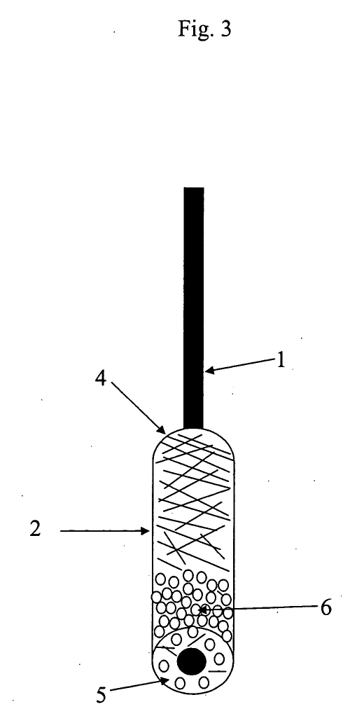

Turning to FIG. 3, the sampling device of FIG. 1 is shown with its hydrophobic cover 3 removed, revealing porous carrier 2 (shown with cross-hatching to denote fibers in the porous material). A portion of porous carrier 2 is impregnated with a labeled binding reagent 6. All of the outermost surface layer (and one or more of any underlying layers) of porous material comprising the entirety of porous carrier 2 may be impregnated with labeled binding reagent 6. However, to minimize background interference and shorten testing time, the impregnation is preferably limited to less than half, and most preferably about 20-40%, of the surface area of the porous material at the distalmost end of porous carrier 2, as indicated in FIG. 3.

Labeled binding reagent 6 is prepared using methods well known in the art. Specific and non-specific (preferably the former) antibodies, antibody fragments, recombinant antibodies, recombinant antibody fragments, antigens, lectins, receptors and/or ligands are suitable binding reagents, which can be attached to any suitable label, such as colored latex, colloidal metals (including gold), dye, fluorescent substances or superparamagnetic particles. Chromogenic substances, particularly fluorochromogens and enzymatic labels, may be used as well. Several different labeled specific binding reagents may be used if the sample is detected for more than one analyte of different binding specificities.

Labeled binding reagent may be applied to porous carrier 2 using tube pumps, which deliver precise volumes of the reagent through a needle or alternatively the porous material can be immersed. The porous material is dried; e.g., in a dry room with a relative humidity less than 20% and further in a dry room with a relative humidity less than 8%. The labeled binding reagent composition and/or porous material may be treated with agents to facilitate release of the binding reagent from the porous material into solution (or, in the case of particulate labeled binding reagents, into suspension) with the sample fluid. Those of ordinary skill in the art will be familiar with releasing agents (such as sugars, casein, and detergents) suitable for use in lateral flow assays that may be utilized in the present invention.

To construct the sampling device, one or more layers of porous material are attached around the distal end of an elongated solid support (made of, for example, wood or plastic). One or more layers of porous material can be attached one at a time or a strip of the porous material can be rolled around the solid support stick. The porous material can be attached by a tape which is first attached to the elongated support, then rolled with the porous material around the stick and finally the tape is attached to itself. Those of ordinary skill in the art will be familiar with alternative means of attaching the porous material to a solid surface, such as inert adhesives and heat bonding. The blocking solution and labeled binding reagent may be applied to porous carrier 2 before or after its attachment to solid support 1.

When completed, the constructed device is dried to a moisture content of 8% or less and packed hermetically separately, or in a kit in combination with a suitable analyzer device.

In a preferred embodiment of the invention, porous carrier 2 is wrapped tightly around solid support 1, but not to the maximum extent possible. Leaving some looseness in the material once it is wrapped around the solid support encourages measured flow of sample fluid from porous carrier 2 once the sampling device is brought into contact with a suitable analyzer device.

For example, viewed from the perspective of total diameter, an exemplary sampling device according to this embodiment of the invention wrapped under 0.1 Nm of torque (within a range of suitable torque preferably ranging from 0.05 to 0.5 Nm) would have a total diameter of 4.7 mm±0.1 mm with the following components present: (a) hydrophobic cover 3 (polyester tape) having a width of 52 mm and a thickness of 0.03 mm; (b) porous carrier 2 (nonwoven polyester) having a width of 27 mm and a thickness of 398 μm; and (c) printed paper having a width of 15 mm and a thickness of 115 μm, all wrapped around (d) solid support 1 (plastic stick) having a thickness of 2 mm. Those of ordinary skill in the art will be able to readily adapt these general dimensions to sampling devices of different sizes constructed according to the invention.

It will be appreciated that the presence of labeled binding reagent on the sampling device of the invention negates any need to use an analyzer device in which further labeled binding reagent is disposed on the test strip or other structure (e.g., a sample application pad) within the analyzer device. As such, the preferred analyzer device for use according to the invention is one which comprises a test strip having at least one test zone (comprised of immobilized binding reagent) and optionally also a control zone.

Those of ordinary skill in the art will be familiar with test strips and methods for immobilizing binding reagents and control reagents thereon for use in lateral flow immunoassays, both in sandwich and competition formats, all of which may be utilized with the sampling device of the present invention, from which labeled binding reagent and sample are supplied.

Thus, an analyzer device suitable for use with the sampling device of the invention may be prepared by immobilizing one or more specific binding reagents and optionally also control reagents directly or indirectly to a porous carrier. The porous carrier can optionally be placed on an impermeable backing or in a housing.

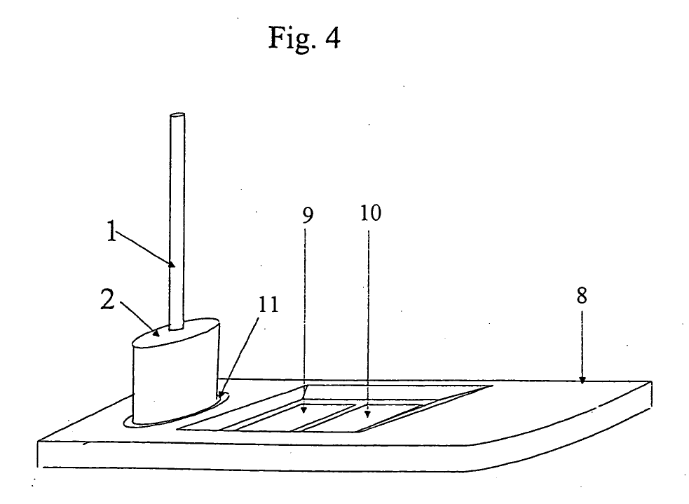

In one embodiment of the invention as shown in FIG. 4, the analyzer device of the invention comprises an impermeable plastic housing 8 in which a test strip (not shown) is disposed. Housing 8 includes a window 9 disposed over a test zone through which test results may be viewed and a window 10 disposed over a control zone through which control results may be viewed. A sample application port 11 is disposed over an portion of the test strip to which no binding reagent is bound, and is provided for insertion therein of the sampling device of the invention.

In another preferred embodiment of the invention a multiple channel analyzer device is used in order to detect the same sample for several analytes at the same time. Every channel of this device can thereto have several detection zones and/or control zones.

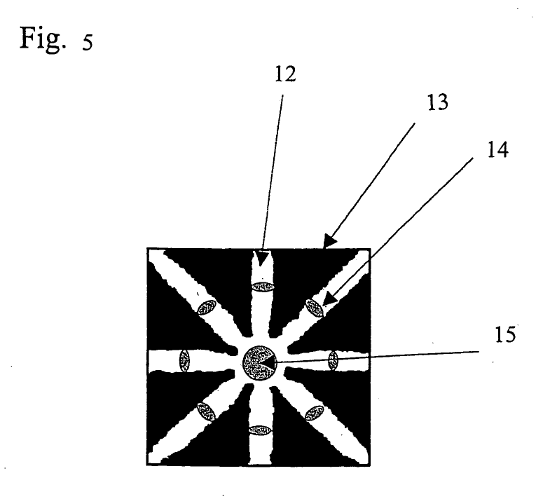

A multiple channel device can be prepared by treating a porous material with a suitable method in order to get different channels for tests of different analytes (or controls). Several analytes can be detected from the same sample by one single test. Thus, in an alternative embodiment of the invention, as shown in FIG. 5, eight channels 12 are made by a suitable method (e.g., blocking, photolithography or etching) on a porous carrier. Channels 12 are separated from each other by areas 13, each of which area is treated to be hydrophobic to prevent flow of sample fluid therethrough. Each channel 13 comprises a test zone or dot 14, comprised of unlabeled binding reagent. Also, a sample application site 15 where the sampling device should be placed into contact with the porous carrier is shown.

The sampling device of the invention can be used by holding the device under the urine stream or by immersing the sampling device in a liquid sample or adding a sample by pipette. If the sampling device is put under the urine stream the preferred time is 2-15 seconds, preferably 5-15 seconds and most preferably 5-10 seconds. If the sampling device is immersed in a liquid sample, the preferred time is 2-30 seconds, preferably 10-20 seconds, most preferably 10-12 seconds. If the liquid sample is added by pipette the preferred amount of sample is 2-40 drops, preferably 5-20 drops and most preferably 5-15 drops. Ten drops is equivalent to about 0.5 ml. During and after sample application, the sampling device is preferably maintained in a substantially vertical orientation, with the wrapped portion of the device oriented downward.

Once sample has been collected, distalmost end 5 of the sampling device (FIG. 3) is placed into contact with the test strip of an analyzer device, allowing sufficient time for the liquid sample alongside with the labeled specific binding reagent or the reaction product thereof to migrate or flow from the diagnostic sampling device to the porous carrier of the analyzer device. The liquid sample and the labeled binding reagent move (e.g., by diffusion or capilliary action) to the test zone of the analyzer device, where the presence or absence of the analyte in the sample fluid is determined.

The embodiments described in the Figures and the Examples are only to be seen as examples of embodiments which are within the scope of the invention. They should not be considered to limit the scope of the invention as defined by the claims.

### EXAMPLE 1

### Pregnancy Test Construction

Example 1 relates to a hCG pregnancy test, in which a protecting impermeable layer is added to the sampling device in order to attach and protect the porous carrier containing the specific labeled reagent. A control zone is added to ensure a proper performance of the test. Components utilized in the device were as follows:

| Component | Raw material | Specifications |
|---|---|---|
| Labeled anti- | In vitro produced, | 0.9% NaCl; |
| hCG antibody | affinity purified | 0.1% NaN₃ as |
| | IgG | preservative |
| Test line: | In vitro produced, | Phosphate-citrate |
| anti-hCG antibody | affinity purified | buffer; NaN₃ as |
| | IgG | preservative |
| Independent | 1. | 1. |
| control system: | keyhole limpet | Copper-containing |
| Labeled | hemocyanin | protein with an |
| 1. keyhole limpet | protein OR | oxygen-carrying |
| hemocyanin OR | 2. | function |
| 2. anti-legumin | in vitro produced, | 2. In PBS buffer |
| IgG | affinity purified | pH 7.4 with |
| | IgG | 0.1% NaN₃ as |
| | | preservative |
| Control line: | In vitro produced, | In sodium phosphate |
| anti-keyhole | affinity purified | buffer with 0.1% |
| limpet hemocyanin | antibodies | NaN₃ as |
| (anti-KLH) OR | | preservative |
| Rabbit or Goat | | (anti-KLH) or |
| anti mouse -IgG | | 0.1 M NaCl with |
| | | 15 mM NaN₃ as |
| | | preservative |
| | | (Rabbit anti |
| | | mouse-IgG)/In |
| | | PBS Buffer |
| | | pH 7.4, with |
| | | 0.02% NaN₃ as |
| | | preservative |
| | | (Goat anti- |
| | | mouse IgG) |
| Membrane | Nitrocellulose | Wicking rate |
| | | 60-200 s/4 cm |
| Gold particles | Gold | Red, diameter |
| | | 20-40 nm |
| Conjugate pad | 100% polyester | 100% polyester |
| (test stick) | | |
| Test strip | Filter | 100% polyester |
| (analyzer device) | | |
| Plastic housing | PVC | Plastic back |
| (analyzer device) | | support |
| Desiccator | Bentonite | |
| Absorbent pad | Special filter | Cellulose |
| | material | acetate |
| Backing plastic | Calendered vinyl | Vinyl |
| | with adhesive | |
| Thin film | Clear polyester | Polyester |
| → At test strip | film | |
| → At swab segment | | |
| Paper label | Cellulose | Pre-printed |
| Sampling stick | 100% Polystyrene | Ø 2.7 mm, |
| | | length 27 mm |

The sampling device of FIG. 1 was constructed using the foregoing materials. The polyester material was first blocked with a blocking solution comprising BSA (0.1-1.0%), Tween 20 (0.01-0.05%) and trehalose (0.5-1.5%). After drying, 3 μl/cm of a gold conjugate solution which comprised a hCG specific binding reagent was applied to the blocked polyester. The solution was added to a 5 mm wide area at the end of the material using tube pumps and was then left to dry in a dry room, with a relative humidity less than 20%. The drying was continued in a dry room with a humidity of less than 8%.

The polyester material was cut in 25 mm×27 mm pieces. Four layers of the porous material was rolled around a round hollow stick made of polypropylene and attached with a tape under a wrapping torque of 0.1 Nm. The tape was first attached to the polypropylene stick and then rolled with the polyester around the stick and finally the tape was attached to itself to form a protective cover over the porous material. As the tape (25 mm×51 mm) was wound around each stick constructed, it was punctured 80-120 times with needles to form vents therein, each having a diameter that varied among the vents between 0.1 and 0.5 mm. The edge with the impregnated labeled specific binding reagent was set in the lower end of the sampling device.

The analyzer device of FIG. 5 was constructed by immobilizing a hCG specific antibody on the porous carrier to form a detection zone. A monoclonal antibody against the labeled specific binding reagent was immobilized on the carrier to form a control zone. The nitrocellulose was then blocked with a blocking solution comprising BSA (0.1-5.0%), TRITON-X-100, BRIJ and saccharose. The material was allowed to dry. The porous carrier was placed in a casing.

The sampling device and the test strip of the analyzer device were dried to 8% moisture content. The test strip (within the casing) and sampling device were packed together hermetically in a hermetic pouch.

The end containing the porous carrier of the sampling device was placed under a urine stream for 10 seconds. Thereafter the sampling device was put in the sample well of the analyzer device. The complex formed of the sample and labeled specific binding reagent was allowed to migrate for 5 minutes after which the result was read in the test window. Red lines were visible in the detection zone of the test window and in the control zone of the control window, which indicated the presence of hCG in the sample.

### EXAMPLE 2

### Pregnancy Test

Example 2 relates to a hCG-pregnancy test where the porous carrier containing the specific labeled reagent is attached without a impermeable layer. A control zone is added to ensure a proper performance of the test.

A polyester filter was pretreated as described in Example 1. One layer of the filter was adhered with an adhesive around a solid stick of wood, and covered with a tape forming a protective layer formed as described in Example 1. The analyzer device was made as in Example 1 and finally the devices were dried and packed as described in Example 1.

The test was performed by immersing the sampling device for 10 seconds in a liquid sample containing hCG. The stick was shaken lightly and thereafter placed in the sample well of the analyzer device. The presence of hCG was detected as described in Example 1.

### EXAMPLE 3

### Fertility Test Detection System

Example 3 describes how the invention can be applied for a fertility test detection system.

3 μl/cm of a gold conjugate solution which comprised three labeled specific antibodies for hCG, LH and FSH was applied to a cellulose filter. The solution was added to a 3 mm wide area at one edge of the material using tube pumps and was then dried. The cellulose filter was blocked with a blocking solution comprising BSA (0.1-1.0%), Tween 20 (0.01-0.05%) and trehalose (0.5-1.5%).

The cellulose filter was cut in 25 mm×27 mm pieces. One piece of the cellulose filter was rolled as four layers around a round hollow stick made of polypropylene and attached with a tape. The edge with the impregnated binding reagent was set in the lower end of the sampling device.

The analyzer device of FIG. 5 was constructed by blocking a porous carrier of nitrocellulose with a blocking solution comprising BSA (0.1-1.0%), Tween 20 (0.01-0.05%) and trehalose (0.5-1.5%). The material was allowed to dry before immobilizing 1 mg/ml of hCG, LH and FSH specific antibodies on the porous carrier to form three different detection zones. 1 mg/ml of a monoclonal antibody against the labeled specific binding reagent was immobilized on the carrier to form a control zone. The porous carrier was dried and placed in a casing.

The test was performed by immersing the end containing the porous carrier of the sampling device in a liquid sample of urine for 10 seconds. The sampling device was then put in the sample well of the analyzer device. The complex formed of the sample and labeled specific binding reagent was allowed to migrate for 5 minutes after which the result was read in the test window. Red lines were visible in the detection zone of the test window and in the control zone of the control window, which indicated the presence of either all or one or more of hCG, LH or FSH in the sample.

### EXAMPLE 4

### Detection System for Venereal Diseases

Example 4 relates to a detection system for HIV_{1,2}.

A polyester filter, used as the porous material of the sampling device was blocked with a blocking solution comprising BSA (0.1-5.0%), TRITON-X-100, BRIJ and saccharose. After drying, 2 μl/cm of a colored latex solution which comprised a polypeptide recognizing both HIV₁ and HIV₂ was applied to the blocked material. The solution was added to a 4 mm area of the filter and the filter was then left to dry in a dry room, with a relative humidity less than 20%.

The polyester filter was cut in 25 mm×27 mm pieces. One piece of the polyester filter was rolled three times around a round hollow stick made of wood and attached with tape (25 mm×51 mm). As the tape was wound around each stick constructed, it was punctured 80-120 times with needles to form vents therein, each having a diameter that varied among the vents between 0.1 and 0.5 mm.

The analyzer device with two test windows and one control window was constructed by immobilizing 0.5 μl of a HIV₁ recombinant antigen and 0.5 μl of a HIV₂ recombinant antigen on the porous carrier to form the detection zones on a porous carrier of nitrocellulose. The material was allowed to dry before the nitrocellulose was blocked with a blocking solution comprising BSA (0.1-5.0%), TRITON-X-100, BRIJ and saccharose. A monoclonal antibody against the labeled specific binding reagent was immobilized on the porous carrier to form a control zone. The porous carrier was dried and placed in a casing. The control zone was placed farthest away from the sample well of the analyzer device.

The test was performed by immersing the end containing the porous carrier of the sampling device in a sample of serum. The sampling device was put in the sample well of the analyzer device. The sample and labeled specific binding reagent was allowed to migrate for 5 minutes after which the result was read in the test windows. The result was read after 10 and 15 minutes as well. As a result red lines were visible in the detection zones of the test window and in the control zone of the control window, which indicated the presence of HIV_{1,2} in the sample.

### EXAMPLE 5

### Multiple Channel Test for Allergens

Example 5 describes use of the invention for allergic testing. The same sample is at the same time tested for several allergens using a multiple channel analyzer device.

A polyester filter, used as the porous material of the sampling device was blocked with a blocking solution comprising BSA (0.1-5.0%), TRITON-X-100, BRIJ and saccharose. After drying, 10 μl of a water solution of colored latex particles coated with anti IgE antibodies recognizing specific IgE molecules was applied to the blocked material. The solution was added to the whole filter and was then left to dry in a dry room, with a relative humidity less than 20%. The polyester filter was cut into 10 mm×20 mm pieces. One piece of the polyester filter was rolled four times around a round hollow stick made of polypropylene attached with tape.

The analyzer device with eight separated channels was constructed by forming the channels in a porous carrier of nitrocellulose and a mylar film 0.5 μl of eight different specific allergens were immobilized on the porous carrier to form the detection zones in each channel of the analyzer device. The material was allowed to dry before the nitrocellulose was blocked with a blocking solution comprising BSA, HEXA and trehalose. The porous carrier was dried.

The test was performed by immersing the distalmost end of the sampling device in a liquid sample of serum. The sample and the labeled specific binding reagent were allowed to migrate downstream in the test strip of the analyzer device for 5 minutes after which the result was read as visible dots in the detection zones, which indicated the presence of allergens in the sample.

1. An immunochemical sampling device for performing an immunoassay comprising: (a) a solid support surrounded by a porous carrier, (b) a labeled binding reagent releasably bound to the porous carrier; and (c) a hydrophobic cover disposed on the porous carrier, wherein the hydrophobic cover comprises pressure equilibrization means, and wherein further the labeled binding reagent is, when mixed with a liquid sample, retained within the porous carrier until the porous carrier is placed in fluid communicative contact with a test strip.

2. The immunochemical sampling device according to claim 1 wherein the pressure equilibrization means comprise vents through the hydrophobic cover.

3. The immunochemical sampling device according to claim 2 wherein the vents are distributed across 10% to 50% of the surface area of the hydrophobic cover.

4. The immunochemical sampling device according to claim 2 wherein the vents each have a diameter from 0.1 to 0.5 mm.

5. The immunochemical sampling device according to claim 2 wherein the hydrophobic cover comprises one or more layers of tape.

6. The immunochemical sampling device according to claim 1 wherein the porous carrier comprises one or more layers of a porous material.

7. The immunochemical sampling device according to claim 6 wherein the porous material is wrapped around the solid support under a wrapping torque from 0.05 and 0.5 Nm.

8. The immunochemical sampling device according to claim 7 wherein the porous material is wrapped around the solid support under a wrapping torque of 0.1 Nm.

9. The immunochemical sampling device according to claim 1 wherein the porous carrier selected from the group consisting of paper, glass fiber, nylon, polyester or cellulose and derivatives thereof.

10. The immunochemical sampling device according to claim 1 wherein the porous material of the sampling device is blocked with a blocking solution comprising natural or synthetic polymers, nonionic detergents or preservation agents.

11. The immunochemical sampling device according to claim 1 wherein the specific binding reagents are selected from the group consisting of antibodies, antigens, lectins, receptors, ligands, fragments thereof or combinations thereof.

12. The immunochemical sampling device according to claim 1 wherein the label is selected from comprising colored latex, gold, metal, dye, fluorogenic substances, superparamagnetic substances, chromogenic substances, fluorochromogens or enzymatic labels.

13. The immunochemical sampling device according to claim 1 wherein the labeled binding reagent is bound to 20% to 40% of the surface area of the distalmost end of the porous carrier.

14. A kit for use in an immunochemical analysis, the kit comprising an immunochemical sampling device according to claim 1 and an analyzer device having at least one binding reagent disposed therein.

15. The kit according to claim 10 wherein the analyzer device comprises a test strip on which a binding reagent is immobilized at a point downstream of a sample application site for contact with the sampling device.

16. The kit according to claim 11 wherein no binding reagent is bound to the test strip at the sample application site.

17. A process for preparing an immunochemical sampling device comprising the steps of
a. wrapping one or more layers of a porous carrier around a solid support and securing the porous carrier thereto, wherein the porous material is comprised of a porous material on which a labeled binding reagent is releasably bound;
b. wrapping the porous carrier in a hydrophobic cover secured thereto, wherein the hydrophobic cover comprises pressure equilibrization means.

18. The process according to claim 16 wherein the pressure equilibrization means comprises vents through the hydrophobic cover.

19. The process according to claim 17 wherein the vents are formed by perforating the hydrophobic cover as it is wrapped around the porous carrier.

20. The process according to claim 16 further comprising the step a′ of disposing the labeled binding reagent onto the porous material so the labeled binding reagent becomes bound to 20% to 40% of the surface area of one or more layers of porous material at the distalmost end of the porous carrier.

21. The process according to claim 16 wherein the porous carrier is wrapped around the solid support under a torque from 0.05 to 0.5 Nm.

22. The process according to claim 21 wherein the torque applied is 0.1 Nm.

23. An immunochemical method for determining the presence or absence of an analyte in a liquid sample comprising the steps of
a. adding the liquid sample to a sampling device according to claim 1, wherein the liquid sample is retained in the sampling device until brought into fluid communicative contact with a test strip;
b. placing the sampling device into fluid communicative contact with a sample application site of a test strip, wherein at least one binding reagent for the analyte is immobilized on the test strip to form a test site downstream of the sample application site; and,
c. allowing sufficient time for the sample and the labeled binding reagent to migrate from the sampling device to the test strip;
d. observing the test result at the test site.
